(19) United States
(12) Patent Application Publication
(10) Pub. No.: US 2022/0087554 A1
(43) Pub. Date: Mar. 24, 2022

(54) DELIVERY DEVICES, SYSTEMS, AND METHODS OF USE FOR POSITIONING AND USING HEMODYNAMIC MONITORING SYSTEMS

(71) Applicant: Shifamed Holdings, LLC, Campbell, CA (US)

(72) Inventors: Amr Salahieh, Saratoga, CA (US); Tom Saul, Moss Beach, CA (US); Brian Fahey, Menlo Park, CA (US); Marwan Berrada, Los Gatos, CA (US)

(21) Appl. No.: 17/600,315

(22) PCT Filed: Apr. 3, 2020
(86) PCT No.: PCT/US2020/026738

§ 371 Date: Sep. 30, 2021

Related U.S. Application Data
(60) Provisional application No. 62/830,313, filed on Apr. 5, 2019.

(51) Int. Cl. A61B 5/0215; A61B 5/02; A61B 5/00

(52) U.S. Cl. CPC A61B 5/02156; A61B 5/746; A61B 5/742; A61B 5/02028

(57) ABSTRACT

The present technology generally relates to hemodynamic monitoring devices, as well as delivery systems adapted for the implantation of implantable pressure sensors or other implantable devices. In some embodiments, for example, the present technology includes a method of implanting a pressure sensing implant in a human patient. The method includes intravascularly advancing a delivery device carrying the pressure sensing implant toward a pulmonary artery of the patient, and sensing pressure in at least one of a right atrium, a right ventricle, and the pulmonary artery of the patient using a pressure sensing device carried by the delivery device. Sensing the pressure occurs before the pressure sensing implant is fully deployed in the pulmonary artery from the delivery device.

### CROSS-REFERENCE TO RELATED APPLICATION(S)

This application claims priority to U.S. Provisional Patent Application No. 62/830,313 filed Apr. 5, 2019, and which is incorporated herein by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### TECHNICAL FIELD

The present technology generally relates to hemodynamic monitoring devices, as well as delivery systems adapted for the implantation of implantable pressure sensors or other implantable devices.

### BACKGROUND

Invasive hemodynamic monitoring devices are typically delivered to the pulmonary artery (“PA”) for the purpose of measuring PA pressure. These devices are primarily used for heart failure and left ventricular assist device (“LVAD”) patients to remotely monitor their internal blood pressure and clinically react to physiological changes that might occur. One such device for measuring and monitoring PA pressure, for example, is manufactured by Abbott Laboratories (Chicago, Ill.) and sold under the trademark CARDIOMEMS. This device is passive in nature (i.e., no battery/internal power source) and reads hemodynamic pressure when excited by a radiofrequency (“RF”) antenna (a so-called “wand”). The pressure sensor then measures blood pressure and sends the information to an outside console.

The manufacturer-recommended process for implantation of a sensor for the conventional device described above (i.e., a CardioMEMS sensor) is a procedure that requires multiple steps and is not fully optimized. Referring to FIG. 1, for example, successful implantation the sensor typically relies in part on a Swan-Ganz (“SG”) catheter 10 as a navigation tool to arrive to the adequate implantation location within a patient. The SG catheter 10 comprises an inflatable balloon 12 that, once inflated with fluid delivered via syringe 14, is allowed to “float” within the vasculature and act as a sail that helps guide navigation of the SG catheter 10 until it reaches a desired location. The SG catheter 10 is instrumented so that the blood pressure in multiple locations and/or cardiac output can be measured (e.g., using thermodilution techniques). Pressure readings can be obtained, for example, by coupling proximal ends of individual lumens to transducers, where the proximal ends are in communication with ports disposed on the catheter body.

FIG. 2 is a flow diagram illustrating a conventional method 20 for delivery and deployment of a CardioMEMS sensor (such as the SG catheter 10 of FIG. 1). After initial insertion of the SG catheter and navigation of the SG catheter to a right atrium (“RA”) of the patient, the method 20 next includes pressure readings taken from the RA (step 24), pressure readings taken from a right ventricle (“RV”) of the patient (step 26), and pressure readings taken from a PA of the patient (step 28) using ports on the SG catheter 10. Current SG catheters do not generally include pressure sensors; rather pressure waveforms are measured by coupling signals through the proximal hubs to a connected external transducer. External transducers are readily available in most relevant clinical settings and the pressure waveform is typically displayed on the screen for the doctors.

In the method of FIG. 2, the block shown in broken lines indicate where one device is exchanged for another, which takes up time during the procedure. For example, at step 30, after a guidewire is placed in the patient, the SG catheter 10 is removed. The method continues at step 31 with a CardioMEMS delivery catheter being advanced over the guidewire, and at step 40 the implantable sensor is positioned and deployed. Once deployed, the method 20 further includes placing a wand in proximity to the patient (step 42) and using the wand to obtain a signal from the implant. The implant catheter is then removed at step 44, and at step 45 the SG catheter 10 is again repositioned and used to measure pulmonary artery wedge pressure at step 48. Cardiac output can be measured at step 50 using thermal dilution and the corresponding proximal port.

Positioning a passive implant like the CardioMEMS sensor requires calibration steps, such as those set forth in the method 20 of FIG. 2. Such calibration allows for implant pressure readings to be calibrated against pressure readings obtained during the implantation procedure. The conventional implantation procedure set forth in FIG. 2, however, also includes using a typical SG catheter with a number of inefficiencies addressed by the technology described herein.

### BRIEF DESCRIPTION OF THE DRAWINGS

FIG. 1 illustrates a conventional device for measuring and monitoring PA pressure in a subject.

FIG. 2 is a flow diagram illustrating a method for delivery and deployment of the device of FIG. 1.

FIG. 3 illustrates delivery device configured to carry, position, and deploy an implantable sensing device in accordance with an embodiment of the present technology.

FIG. 4 is a flow diagram illustrating a method for delivery and deployment of the device of FIG. 3.

FIG. 5 is a display diagram of pressure readouts correlated with sensor position.

### DETAILED DESCRIPTION

The present technology is generally related to hemodynamic monitoring devices, as well as delivery systems adapted for the implantation of implantable pressure sensors or other implantable devices. In one embodiment of the present technology, for example, a method of implanting a pressure sensing implant in a pulmonary artery comprises intravascularly advancing the pressure sensing implant on a pathway towards a pulmonary artery. The method can include sensing pressure (or data/information indicative of pressure) in at least one of a RA, RV, and PA of the patient via a pressure sensing device carried by the pressure sensing implant. Such pressure measurements occur before the pressure sensing implant is fully deployed in the pulmonary artery using the delivery device. In some embodiments, the method can also include automatically determining the location (e.g., RA, RV, PA, wedge) of the pressure sensing device based on at least one type of sensed data from the pressure sensing device (e.g., data indicative of a pressure waveform associated with the particular location).

The implantable components described herein can be temporary or permanent. For example, in some embodiments the implantable can be removable using known techniques. In some embodiments, aspects of the implantable components can be biodegradable. In some embodiments, the implantable components are configured to remain implanted for a prolonged period of time (e.g., at least one month, at least three months, at least one year, etc.).

For example, some embodiments of the present technology relate to active pressure measurement devices configured and adapted for implantation in the PA of a patient. Active pressure measurement refers to a pressure sensing implant with an on-board power-source (e.g., battery), and an onboard chip and architecture that allows communication with an external device (i.e., device located outside a body of the patient). The communication can be through RF band signals, for example using Bluetooth or some other suitable protocol. The devices can be periodically “awoken” (e.g., every 1 hour, every 2 hours, or at timed intervals set by a clinical provider) from a low-power sleep state in order to measure one or more physiological signals, and can be adapted to process the signals via an ultra-low power computing chip.

Some aspects of this disclosure may be directed to diagnostics related to heart failure. For example, some aspects of the disclosure are related to communicating patient data and/or information related to heart failure to one or more devices and/or systems so that one or more individuals (e.g., a nurse, physician, patient, etc.) can gain access to the data and/or information. Increased compliance with best standard practices and treatments for addressing heart failure can greatly improves a patient's health. Accordingly, as provided herein, some aspects of the disclosure are related to increasing compliance with a heart failure treatment plan. Aspects of this disclosure may also reduce the costs for care providers by one or more of the following: improving patient monitoring (e.g. improving the amount and quality of patient data being obtained), reducing patient hospitalizations, and/or reducing the cost of monitoring by flagging non-compliant patients and focusing costs and efforts on needy patients.

The disclosure herein sets forth a variety of devices and systems that can be used to perform or accomplish one or more of the functions herein. The devices and systems are exemplary and are not necessarily limiting. The devices may include aspects, elements, or features that are not necessarily needed to accomplish or perform all functions herein. Suitable device and/or system features from different embodiments and figures can be combined unless indicated herein to the contrary.

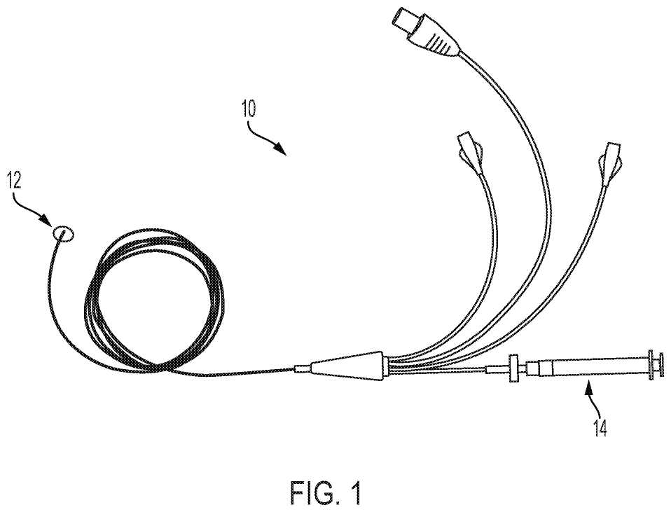

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1-5.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

### A. Select Embodiments of Delivery Devices for Implantable Sensing Devices and Associated Methods

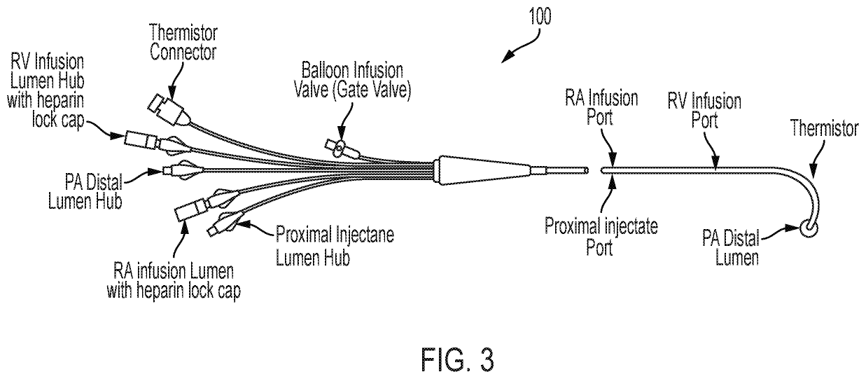

FIG. 3 illustrates a delivery device 100 configured in accordance with an embodiment of the present technology. The delivery device 100 is configured to carry, position, and deploy an implantable sensing device, as well as provide one or more existing functions of conventional SG catheters. For example, as set forth above, conventional SG catheters don't generally include built-in pressure sensors, and pressure waveforms are measured through a proximal hub/port that may be connected to an external transducer. After a physician views the pressure waveforms from the SG catheter, the implant is then positioned using a separate delivery device, which adds time and complexity to the overall implant procedure.

In contrast with such conventional devices and procedures, the delivery device 100 is adapted to carry and deploy the implantable sensing device and includes integrated pressure sensing capabilities therein. For example, the delivery device 100 is configured to sense the right atrium, right ventricle, pulmonary artery, and pulmonary artery wedge pressures without requiring the use of the SG ports and external transducers as is commonly done with conventional SG catheters.

In some embodiments, the delivery device 100 may comprise a thermistor connector. The thermistor connector is adapted for thermal dilution and may include active electrical components, such as leads that carry electrical power and signals back to a main terminal (not shown) used by the clinician or operator. The pulmonary artery wedge pressure could be either manually entered or automatically stored into the chip.

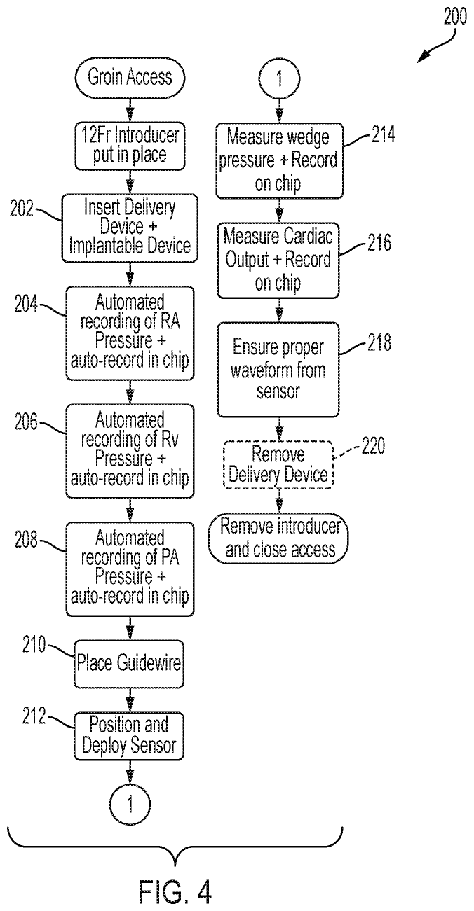

FIG. 4 is a flow diagram illustrating a method 200 of operating the delivery device 100 of FIG. 3 in accordance with various embodiments of the present technology. The method 200 is illustrated as a set of steps, operations, or processes. In some embodiments, one or more of the steps may be implemented, at least in part, in the form of executable code or instructions stored on non-transitory, tangible, machine-readable media. All or a subset of the steps of the method 200 can be executed at least in part by various components or features of a delivery device, such as any of the delivery devices described herein. Additionally, or alternatively, all or a subset of the steps of the method 200 can be executed at least in part by an operator (e.g., a physician, a clinician, a user, etc.) of the delivery device.

Referring to FIGS. 3 and 4 together, the method 200 begins at step 202 with insertion of the delivery device 100 carrying the implantable device in a patient. After insertion, the method 200 with navigation of a distal region of the delivery device 100 to a RA of the patient. At step 204, right atrial pressure is sensed using the pressure sensor in/on the implantable device and automatically recorded in the on-board chip of the implantable device.

At step 206, right ventricle pressure is sensed using the pressure sensor in/on the implantable device and automatically recorded in the on-board chip of the implantable device. The method 200 continues at step 208 with sensing of pulmonary artery pressure using the pressure sensor in/on the implantable device and automatically recording the sensed pressure in the on-board chip of the implantable device. At step 210, a guidewire can be placed, followed by positioning deployment of the implant at step 212.

The method 200 continues at step 214 with measuring and recording pulmonary artery wedge pressure. At step 216, cardiac output can then be measured using the same technique as is done on a typical SG catheter (at least some of the functionality for which is integrated into the delivery device 100 of FIG. 3), and recorded on the implant chip. The method 200 further includes, communicating the pressure waveform to an external device (e.g., via Bluetooth or another suitable communication protocol) to ensure the proper waveform is detected (step 218).

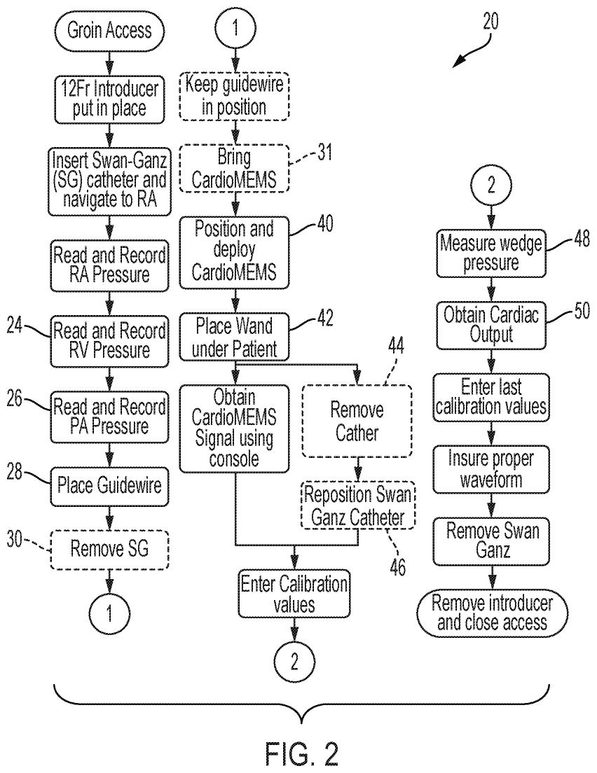

At step 220, the delivery device 100 is removed, which in the method 200 of FIG. 4 is the only time the delivery device 100 needs to be removed prior to the completion of the process. As mentioned previously, the method 200 is expected to be considerably more efficient and reduce procedure time as compared with conventional methods (such as that described above with reference to FIG. 2), which involve multiple tool changes and removals.

One additional advantage of using the delivery device 100 in association with the method 200 of FIG. 4 is that the delivery device 100 is adapted to directly take measurements from the implant as the implant is advanced into position. In contrast, the conventional procedure 20 of FIG. 2 requires a number of additional calibration steps.

More specifically, in the method 200 of FIG. 4 (and with reference to the delivery device 100 of FIG. 3), a pressure sensor from the hemodynamic implant on or carried by the delivery device 100 directly communicates its results to an on-board chip and does not require any operator intervention. The pressure sensor on the delivery device is activated before insertion in the body (i.e., before or as a part of step 202) and acts as a live-pressure sensor on a catheter. As the delivery catheter is navigated to the PA, the sensor is actively measuring pressure in the RA, RV, PA, and PAWP (FIG. 4) of the patient. With special instructions or programming stored on the on-board chip (e.g., software adapted to recognize the different locations waveforms), the onboard computing system can be configured or trained to recognize the pressure waveform at different locations (e.g., RA or RV, etc.) and actively calculate, validate, and store the necessary parameters for an auto-calibration of the device as it is navigated to the PA location. This type of auto-calibration is expected to save time and reduce the required number of operator actions compared to the process 20 of FIG. 2.

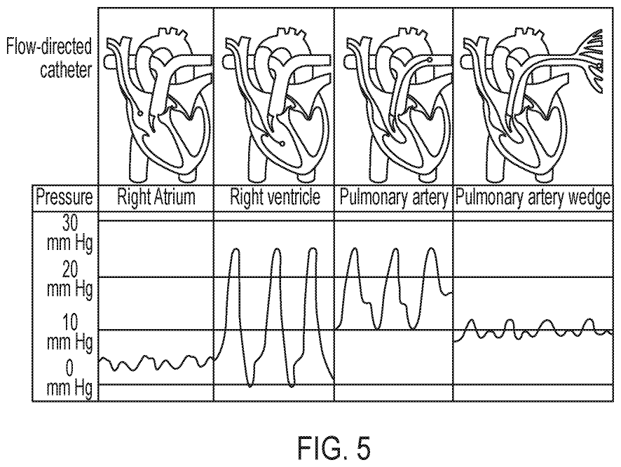

The delivery device 100 may also comprise an indicator element (e.g., visual, audio, tactile) adapted to provide indication to the clinician that a measured pressure at a certain anatomical location was properly acquired. The indicator element may be positioned, for example, on the hub or another suitable external location of the delivery device 100. By way of example, the indicator element may comprise a light emitting diode (LED) configured to display red, green, or yellow light (or another suitable visual indicator) to provide status updates to the clinician during operation. In one particular embodiment, for example, if the RA-LED is red and the physician has moved to the RV, the physician can return the device to the RA and measure the pressure. Because the pressure waveforms in the different locations (RA, RV, PA, wedge) are very distinct (see, e.g., the pressure readouts shown in FIG. 5 that are correlated with the sensor position), the on-board software/algorithm can easily infer its location based on the sensed pressure. As part of any process herein, any or all pressure values can be reviewed and accepted by the clinician before continuing the implant procedure and permanently implanting the device in the PA.

One advantage of embodiments of the present technology is that, to accelerate the calibration process, the hemodynamic monitoring devices disclosed herein may comprise an independent pressure sensor on the corresponding delivery catheter in addition to the sensor on the device itself. Thus, the hemodynamic monitoring devices do not require any calibration using external input parameters since the device is adapted and configured with auto-calibration functionality using the sensed data from the implant.

### EXAMPLES

Several aspects of the present technology are set forth in the following examples:
1. A method of implanting a pressure sensing implant in a pulmonary artery, the method comprising:
intravascularly advancing the pressure sensing implant on a pathway towards a pulmonary artery; and
sensing pressure (or data/information indicative of pressure) in at least one of a right atrium, a right ventricle, and a pulmonary artery using a pressure sensing device carried by the pressure sensing implant,
wherein the sensing pressure occurs before the pressure sensing implant is fully deployed in the pulmonary artery from a delivery device.
2. The method of example 1 wherein sensing pressure comprises sensing pressure in the right atrium, the right ventricle, and the pulmonary artery using the pressure sensing device carried by the pressure sensing implant.
3. The method of example 1 or example 2, further comprising activating the pressure sensing device from a deactivated state before advancing the pressure sensing implant into the right atrium, and optionally while the pressure sensing implant is disposed external to the patient.
4. The method of any examples 1-3 wherein the sensing pressure comprises continuously sensing pressure with the pressure sensing device.
5. The method of any of examples 1-4, further comprising automatically determining the location (e.g., RA, RV, PA, wedge) of the pressure sensing device based on at least one type of sensed data from the pressure sensing device (e.g., data indicative of a pressure waveform), optionally performed with one or more algorithms stored on a memory device disposed in the pressure sensing implant.
6. The method of any of examples 1-5 wherein automatically determining the location of the pressure sensing device is performed with one or more algorithms adapted to automatically recognize a location based on the at least one type of sensed data.
7. The method of any of examples 1-6, further comprising automatically calibrating one or more processing components disposed in the pressure sensing implant, optionally using at least one algorithm that is adapted to associate a location (e.g., RA, RV) with at least one type of sensed data.
8. The method of any of examples 1-7, further comprising displaying an alert (e.g., visual, audio, tactile) if a pressure reading and/or sensed data from one or more locations (e.g., RA, RV) was not properly sensed or processed, optionally wherein displaying the alert is initiated by an algorithm stored in a memory device, the algorithm configured to automatically detect if a pressure reading and/or sensed data was not properly sensed or processed.
9. The method of any of examples 1-8 wherein intravascularly advancing the pressure sensing implant on a pathway towards a pulmonary artery comprises intravascularly advancing the pressure sensing implant while carried by the delivery device.
10. The method of any of examples 1-9 wherein the implant is deployed in the pulmonary artery without removing any type of delivery catheter or any delivery device (including the delivery device) before deploying the implant from the pulmonary artery.
11. The method of any of examples 1-10 wherein sensed pressure (or data/information indicative thereof) is automatically recorded in a memory device disposed in the pressure sensing implant.
12. The method of any of examples 1-11 wherein intravascularly advancing the pressure sensing implant comprising advancing an active (on-board power supply) pressure sensing implant.
13. The method of any of examples 1-13 wherein the method does not include calibrating the implant using a proximal port coupled to an external transducer.
14. A method of implanting a pressure sensing implant in a pulmonary artery without removing a previously-inserted catheter prior to final deployment of the pressure sensing implant.
15. A pressure sensing implant delivery device, comprising
an elongate body;
an inflatable member carried by the elongate body, wherein the inflatable member is in communication with an inflation lumen in the elongate body and an inflation port positioned to remain outside a patient; and
a pressure sensing implant receiving area.
16. The delivery device of example 15, further comprising a thermistor connection/port.
17. The delivery device of example 15, further comprising one or more infusion lumens.
18. A computer-readable storage medium storing instructions that, when executed by a computing system, cause the computing system to perform operations comprising:
receiving, as input, information indicative of data sensed by a pressure sensing device carried by a pressure sensing implant that is being advanced toward a pulmonary artery of a human patient; and
comparing the input to stored information to automatically determine a location of the pressure sensing device when it sensed the data.
19. The computer-readable storage medium of example 18, further comprising storing the sensed data, and associating the stored data with a location within the patient, optionally one of a right atrium, a right ventricle, and a pulmonary artery of the patient.
20. The computer-readable storage medium of example 18, further comprising automatically calibrating the pressure sensing implant with the stored data.
21. The computer-readable storage medium of example 18, further comprising automatically determining if the sensed data was not properly sensed and/or processed, and optionally providing an alert (e.g., visual, audio, tactile) if it was not.
22. A method of implanting a pressure sensing implant in a pulmonary artery of a human patient, the method comprising:
intravascularly advancing a delivery device carrying the pressure sensing implant toward the pulmonary artery of the patient; and
sensing pressure in at least one of a right atrium, a right ventricle, and the pulmonary artery of the patient using a pressure sensing device carried by the delivery device,
wherein sensing the pressure occurs before the pressure sensing implant is fully deployed in the pulmonary artery from the delivery device.
23. The method of example 22 wherein the pressure sensing implant is configured to be calibrated using sensed data from the delivery device during implantation and does not require calibration using external input parameters.
24. The method of example 22 wherein the pressure sensing implant is configured to be calibrated without being operably coupled to an external transducer during the implantation procedure.
25. The method of example 22, further comprising storing the sensed pressure data, and associating the stored pressure data with one of the right atrium, the right ventricle, and the pulmonary artery within the patient.
26. The method of example 25, further comprising automatically calibrating the pressure sensing implant with the stored data.
27. The method of example 22, further comprising automatically determining the location of the pressure sensing device based on at least one type of sensed data from the pressure sensing device.
28. The method of example 22 wherein the at least one type of sensed data comprises data indicative of a pressure waveform associated with the particular location.
29. The method of example 28 wherein the pressure waveform associated with the particular location comprise the pressure waveform associated with the right atrium, right ventricle, and/or pulmonary artery of the patient.
30. The method of example 22, further comprising activating the pressure sensing device from a deactivated state before intravascularly advancing a delivery device carrying the pressure sensing implant toward the pulmonary artery.
31. The method of example 22, further comprising activating the pressure sensing device from a deactivated state while the pressure sensing implant is external to the patient.
32. The method of example 22 wherein sensing pressure comprises continuously sensing pressure with the pressure sensing device during the implantation procedure.
33. The method of example 22, further comprising automatically calibrating one or more processing components disposed in the pressure sensing implant based, at least in part, on at least one type of sensed data.
34. The method of example 22, further comprising automatically recording sensed pressure data in a memory disposed in the pressure sensing implant.
35. The method of example 22, further comprising displaying an alert if sensed pressure data from one or more locations in the patient was not properly sensed or processed.
36. The method of example 35 wherein displaying the alert comprises displaying a visual indication via an indicator element carried by the delivery device and external to the patient.
37. A computer-readable storage medium storing instructions that, when executed by a computing system, cause the computing system to perform operations comprising:
receiving, as input, information indicative of data sensed by a pressure sensing device carried by a pressure sensing implant that is being advanced toward a pulmonary artery of a human patient; and
comparing the input to stored information to automatically determine a location of the pressure sensing device when it sensed the data.
38. The computer-readable storage medium of example 37, further comprising storing the sensed data and associating the stored data with a right atrium, a right ventricle, and/or a pulmonary artery or the patient.
39. The computer-readable storage medium of example 38, further comprising automatically calibrating the pressure sensing implant with the stored sensed data.
40. The computer-readable storage medium of example 38, further comprising automatically determining if the sensed data was properly sensed and/or processed and, if not properly sensed, providing an alert to a clinician.
41. A method of implanting a pressure sensing implant in a human patient, the method comprising:
intravascularly advancing a delivery device carrying the pressure sensing implant toward a cardiac chamber of the patient; and
sensing pressure in at least one of a right atrium, a right ventricle, and the pulmonary artery of the patient using a pressure sensing device carried by the delivery device,
wherein sensing the pressure occurs before the pressure sensing implant is fully deployed in the cardiac chamber from the delivery device.
42. The method of example 41 wherein the pressure sensing implant is configured to be calibrated using sensed data from the delivery device during implantation and does not require calibration using external input parameters.

### Conclusion

As described above, embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, or other protocols known in the art; energy harvesting means, for example a coil or antenna that is capable of receiving and/or reading an externally-provided signal which may be used to power the device, charge a battery, initiate a reading from a sensor, or for other purposes. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe apparatuses implanted within certain parts of the body, it should be appreciated that similar embodiments could be utilized for apparatuses implanted in or positioned at a variety of other regions of the body.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A method of implanting a pressure sensing implant in a pulmonary artery of a human patient, the method comprising:
intravascularly advancing a delivery device carrying the pressure sensing implant toward the pulmonary artery of the patient; and
sensing pressure in at least one of a right atrium, a right ventricle, and the pulmonary artery of the patient using a pressure sensing device carried by the delivery device,
wherein sensing the pressure occurs before the pressure sensing implant is fully deployed in the pulmonary artery from the delivery device.

2. The method of claim 1 wherein the pressure sensing implant is configured to be calibrated using sensed data from the delivery device during implantation and does not require calibration using external input parameters.

3. The method of claim 1 wherein the pressure sensing implant is configured to be calibrated without being operably coupled to an external transducer during the implantation procedure.

4. The method of claim 1, further comprising storing the sensed pressure data, and associating the stored pressure data with one of the right atrium, the right ventricle, and the pulmonary artery within the patient.

5. The method of claim 4, further comprising automatically calibrating the pressure sensing implant with the stored data.

6. The method of claim 1, further comprising automatically determining the location of the pressure sensing device based on at least one type of sensed data from the pressure sensing device.

7. The method of claim 1 wherein the at least one type of sensed data comprises data indicative of a pressure waveform associated with the particular location.

8. The method of claim 7 wherein the pressure waveform associated with the particular location comprise the pressure waveform associated with the right atrium, right ventricle, and/or pulmonary artery of the patient.

9. The method of claim 1, further comprising activating the pressure sensing device from a deactivated state before intravascularly advancing a delivery device carrying the pressure sensing implant toward the pulmonary artery.

10. The method of claim 1, further comprising activating the pressure sensing device from a deactivated state while the pressure sensing implant is external to the patient.

11. The method of claim 1 wherein sensing pressure comprises continuously sensing pressure with the pressure sensing device during the implantation procedure.

12. The method of claim 1, further comprising automatically calibrating one or more processing components disposed in the pressure sensing implant based, at least in part, on at least one type of sensed data.

13. The method of claim 1, further comprising automatically recording sensed pressure data in a memory disposed in the pressure sensing implant.

14. The method of claim 1, further comprising displaying an alert if sensed pressure data from one or more locations in the patient was not properly sensed or processed.

15. The method of claim 14 wherein displaying the alert comprises displaying a visual indication via an indicator element carried by the delivery device and external to the patient.

16. A computer-readable storage medium storing instructions that, when executed by a computing system, cause the computing system to perform operations comprising:
receiving, as input, information indicative of data sensed by a pressure sensing device carried by a pressure sensing implant that is being advanced toward a pulmonary artery of a human patient; and
comparing the input to stored information to automatically determine a location of the pressure sensing device when it sensed the data.

17. The computer-readable storage medium of claim 16, further comprising storing the sensed data and associating the stored data with a right atrium, a right ventricle, and/or a pulmonary artery or the patient.

18. The computer-readable storage medium of claim 17, further comprising automatically calibrating the pressure sensing implant with the stored sensed data.

19. The computer-readable storage medium of claim 17, further comprising automatically determining if the sensed data was properly sensed and/or processed and, if not properly sensed, providing an alert to a clinician.

20. A method of implanting a pressure sensing implant in a human patient, the method comprising:
intravascularly advancing a delivery device carrying the pressure sensing implant toward a cardiac chamber of the patient; and
sensing pressure in at least one of a right atrium, a right ventricle, and the pulmonary artery of the patient using a pressure sensing device carried by the delivery device,
wherein sensing the pressure occurs before the pressure sensing implant is fully deployed in the cardiac chamber from the delivery device.

21. The method of claim 20 wherein the pressure sensing implant is configured to be calibrated using sensed data from the delivery device during implantation and does not require calibration using external input parameters.
